SEQUENCE LISTING (29 sequences)

SEQ ID NO: 1 - DNA (cDNA), 18 bp; single-stranded, linear
  CDS 1..18
tacttgttgccgcgcagg

SEQ ID NO: 2 - protein, 6 aa
YLLPRR

SEQ ID NO: 3 - protein, 6 aa
  Modified-site one-of(1): /note= “Xaa represents Tyr, Asp, Glu, Leu or Ile”
  Modified-site one-of(2): /note= “Xaa represents Leu, Cys, Asp or Glu”
  Modified-site one-of(3): /note= “Xaa represents Leu or Val”
  Modified-site one-of(4): /note= “Xaa represents Pro or Met”
  Modified-site one-of(5): /note= “Xaa represents Arg, Asp, Glu, Pro, Cys, Phe, Ile, Asn, Gln or Met”
  Modified-site one-of(6): /note= “Xaa represents Arg, Asp, Glu, Ser or Cys”
XXXXXX

SEQ ID NO: 4 - DNA (cDNA), 51 bp; single-stranded, linear
  CDS 1..51: /note= “Nucleotides 1-3 are of non-HCV origin”
tgccagatcgttggtggagtttacttgttgccgcgcaggggccctagattg

SEQ ID NO: 5 - protein, 17 aa
CQIVGGVYLLPRRGPRL

SEQ ID NO: 6 - DNA (cDNA), 51 bp; single-stranded, linear
  CDS 1..51: /note= “Nucleotides 49 to 51 are of non-HCV origin”
cagatcgttggtggagtttacttgttgccgcgcaggggccctagattgtgc

SEQ ID NO: 7 - protein, 17 aa
QIVGGVYLLPRRGPRLC

SEQ ID NO: 8 - protein, 97 aa
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWL

SEQ ID NO: 9 - protein, 8 aa
GVYLLPRR

SEQ ID NO: 10 - protein, 8 aa
VYLLPRRG

SEQ ID NO: 11 - protein, 8 aa
YLLPRRGP

SEQ ID NO: 12 - protein, 8 aa
GGGQIVGG

SEQ ID NO: 13 - protein, 8 aa
GGQIVGGV

SEQ ID NO: 14 - protein, 8 aa
GQIVGGVY

SEQ ID NO: 15 - protein, 8 aa
QIVGGVYL

SEQ ID NO: 16 - protein, 8 aa
IVGGVYLL

SEQ ID NO: 17 - protein, 8 aa
VGGVYLLP

SEQ ID NO: 18 - protein, 8 aa
  Region 4..8: /note= “Immunodominant region”
GGVYLLPR

SEQ ID NO: 19 - protein, 8 aa
  Region 3..8: /note= “Immunodominant region”
GVYLLPRR

SEQ ID NO: 20 - protein, 8 aa
  Region 2..7: /note= “Immunodominant region”
VYLLPRRG

SEQ ID NO: 21 - protein, 8 aa
  Region 1..6: /note= “Immunodominant region”
YLLPRRGP

SEQ ID NO: 22 - protein, 8 aa
LLPRRGPR

SEQ ID NO: 23 - protein, 8 aa
LPRRGPRL

SEQ ID NO: 24 - protein, 8 aa
PRRGPRLG

SEQ ID NO: 25 - protein, 8 aa
RRGPRLGV

SEQ ID NO: 26 - protein, 8 aa
RGPRLGVR

SEQ ID NO: 27 - protein, 8 aa
GPRLGVRA

SEQ ID NO: 28 - protein, 8 aa
PRLGVRAT

SEQ ID NO: 29 - protein, 8 aa
RLGVRATR

(19) United States
(12) Patent Application Publication
(10) Pub. No.: US 2002/0150990 A1
(43) Pub. Date: Oct. 17, 2002

(54) Hepatitis C virus peptides

(76) Inventor: Ian Pike, Bromley (GB)

Correspondence Address: Nixon & Vanderhye P.C., 8th Floor, 1100 N. Glebe Rd.,, Arlington, VA, 22201, US

(21) Appl. No.: 09/756,875

(22) Filed: Jan. 10, 2001

Related U.S. Application Data
(63) Continuation of application No. 08/842,114, filed on Apr. 28, 1997, now abandoned.
Application No. 08/842,114 is a continuation of application No. 08/295,721, filed on Aug. 29, 1994, now abandoned.
Application No. 08/295,721 is a 371 of international application No. PCT/GB93/00410, filed on Feb. 26, 1993.

(30) Foreign Application Priority Data
Feb. 28, 1992 (GB) 9204274-6

(51) Int. Cl. C12P 21/02; C12N 5/06; C12N 15/86

(52) U.S. Cl. 435/69.3; 435/456; 435/320.1; 435/325; 536/23.72; 530/329

(57) ABSTRACT

This invention relates to a novel peptide, having the sequence as shown in SEQ ID NO's 1 and 2, capable of binding to an antibody specific for parenterally transmitted non-A non-B hepatitis (PT-NAN BH), in particular (HCV) and the use of such a peptide in an immunoassay for the detection of HCV or a vaccine for its prevention.

This invention relates to a novel peptide capable of binding to antibody specific for parenterally transmitted non A non B hepatitis (PT-NANBH) in particular (HCV) and the use of such a peptide in an immunoassay for the detection of HCV or a vaccine for its prevention.

Non A non B hepatitis (NANBH) is by definition a diagnosis of exclusion and has generally been employed to describe cases of viral hepatitis infection in human beings that are not due to hepatitis A or B viruses. The PT-NANBH virus has also been referred to as Hepatitis C Virus (HCV). In the majority of such cases, the cause of the infection has not been identified although, on clinical and epidemiological grounds, a number of agents have been thought to be responsible as reviewed in Shih et al (Prog Liver Dis., 1986, 8, 433-452). In the USA alone, up to 10% of blood transfusions can result in NANBH which makes it a significant problem.

GB-A-2 239 245 discloses nucleotide and polypeptide sequences of a viral agent responsible for Post Transfusional NANBH (PT-NANBH), the core region of this viral agent responsible for PT-NANBH is disclosed within the nucleotide and peptide sequences. The core region of the disclosed viral agent responsible for PT-NANBH was not analysed in GB-A-2 239 245. Substantial portions of the core region were used in recombinant polypeptides namely BHC-11 as disclosed in GB-A-2 239 245.

Substantial regions of the core region of the viral agent responsible for PT-NANBH disclosed in GB-A-2 239 245, were also used to detect HCV infection in British Application No. 9203803.3 filed in the name of Wellcome Foundation Limited on Feb. 21, 1992.

EP-A-0 445 523 discloses an assay for detecting the presence of an antibody to an HCV antigen by contacting the sample with a polypeptide containing at least one epitope of an HCV antigen. A polypeptide from the putative core region of HCV is used. Specific epitopic regions of the polypeptides are not identical.

It has surprisingly been found by the present inventors that several domains within the N-terminal region of the HCV core protein are epitopic. It has also surprisingly been found that of the sera reactive with N-terminal region of the core protein the majority react with a single domain exclusively or in combination with any of the other epitopic domains. This single domain has been termed the immunodominant epitope. This immunodominant epitope has been isolated by the present inventors.

Accordingly, the present invention provides a HCV viral peptide having the following sequence (SEQ ID NO's: 1 and 2):

Tyr-Leu-Leu-Pro-Arg-Arg (YLLPRR)

This sequence may be found at residues 35-40 of the core protein. The present invention also provides a DNA sequence encoding the peptide as defined above. The DNA sequence may be synthetic or cloned. Preferably the DNA sequence is as set forth in SEQ ID NO: 1.

A novel synthetic peptide has also been synthesized which peptide has improved assay sensitivity and specificity, when compared to the natural peptide as disclosed above, in assays for HCV.

Accordingly, the present invention also provides a peptide of the general formula I

X₁-X₂-X₃-X₄-X₅X₆ (SEQ ID NO: 3) I

wherein;

X₁ represents Tyrosine, Aspartic Acid, Glutamic Acid, Leucine or Isoleucine;

X₂ represents Leucine, Cysteine, Aspartic Acid or Glutamic Acid;

X₃ represents Leucine, Valine;

X₄ represents Proline or Methionine;

X₅ represents Aspartic Acid, Glutamic Acid, Proline, Cysteine, Phenylalanine, Isoleucine, Asparagine, Glutamine or Methionine; and

X₆ represents Arginine, Aspartic Acid, Glutamic Acid, Serine or Cysteine

and wherein the peptide is not of the formula Tyr-Leu-Leu-Pro-Arg-Arg (SEQ ID NO: 1 and 2).

The present invention also provides a DNA sequence encoding the peptide of general formula I, which sequence can be readily ascertained, bearing in mind the degeneracy of the genetic code and codon usage.

The peptides of the present invention may be used individually, as part of a larger polypeptide and/or as a mixture of peptides with other peptides from the HCV genome, a heterologous genome or topographically related peptides.

The present invention also provides peptides which comprise the peptides as defined above, namely, SEQ ID NO's: 4 and 5 and SEQ ID NO's: 6 and 7.

The present invention also provides DNA sequences encoding the peptide as set forth above. Preferably the DNA sequences are as set forth in SEQ ID NO's: 4 and 6.

The present invention also provides expression vectors containing the DNA sequences as herein defined, which vectors being capable, in an appropriate host, of expressing the DNA sequence to produce the peptides as defined herein.

The expression vector normally contains control elements of DNA that effect expression of the DNA sequence in an appropriate host. These elements may vary according to the host but usually include a promoter, ribosome binding site, translational start and stop sites, and a transcriptional termination site. Examples of such vectors include plasmids and viruses. Expression vectors of the present invention encompass both extrachromosomal vectors and vectors that are integrated into the host cell's chromosome. For use in E. coli, the expression vector may contain the DNA sequence of the present invention optionally as a fusion linked to either the 5′- or 3′-end of the DNA sequence encoding, for example, β-galactosidase or to the 3′-end of the DNA sequence encoding, for example, the trip E gene. For use in the insect baculovirus (AcNPV) system, the DNA sequence is optionally fused to the polyhedrin coding sequence.

The present invention also provides a host cell transformed with expression vectors as herein defined.

Examples of host cells of use with the present invention include prokaryotic and eukaryotic cells, such as bacterial, yeast, mammalian and insect cells. Particular examples of such cells are E. coli, S. cerevisiae, P. pastoris, Chinese hamster ovary and mouse cells, and Spodoptera frugiperda and Tricoplusia ni. The choice of host cell may depend on a number of factors but, if post-translation modification of the HCV viral peptide is important, then an eukaryotic host would be preferred.

The present invention also provides a process for preparing a peptides as defined herein which comprises isolating the DNA sequence, as herein defined, from the HCV genome, or synthesising DNA sequence encoding the peptides as defined herein, or generating a DNA sequence encoding the peptide of Formula I, inserting the DNA sequence into an expression vector such that it is capable, in an appropriate host, of being expressed, transforming an host cells with the expression vector, culturing the transformed host cells, and isolating the peptide.

The DNA sequence encoding the peptide may be synthesized using standard procedures (Gait, Oligonucleotide Synthesis: A Practical Approach, 1984, Oxford, IRL Press).

The desired DNA sequence obtained as described above may be inserted into an expression vector using known and standard techniques. The expression vector is normally cut using restriction enzymes and the DNA sequence inserted using blunt-end or staggered-end ligation. The cut is usually made at a restriction site in a convenient position in the expression vector such that, once inserted, the DNA sequences are under the control of the functional elements of DNA that effect its expression.

Transformation of an host cell may be carried out using standard techniques. Some phenotypic marker is usually employed to distinguish between the transformants that have successfully taken up the expression vector and those that have not. Culturing of the transformed host cell and isolation of the peptide as required may also be carried out using standard techniques.

The peptides of the present invention may be prepared by synthetic methods or by recombinant DNA technology. The peptides are preferably synthesized using automatic synthesizers.

Antibody specific to a peptide of the present invention can be raised using the peptide. The antibody may be polyclonal or monoclonal. The antibody may used in quality control testing of batches of the peptides; purification of a peptide or viral lysate; epitope mapping; when labelled, as a conjugate in a competitive type assay, for antibody detection; and in antigen detection assays.

Polyclonal antibody against a peptide of the present invention may be obtained by injecting a peptide, optionally coupled to a carrier to promote an immune response, into a mammalian host, such as a mouse, rat, sheep or rabbit, and recovering the antibody thus produced. The peptide is generally administered in the form of an injectable formulation in which the peptide is admixed with a physiologically acceptable diluent. Adjuvants, such as Freund's complete adjuvant (FCA) or Freund's incomplete adjuvant (FIA), may be included in the formulation. The formulation is normally injected into the host over a suitable period of time, plasma samples being taken at appropriate intervals for assay for anti-HCV viral antibody. When an appropriate level of activity is obtained, the host is bled. Antibody is then extracted and purified from the blood plasma using standard procedures, for example, by protein A or ion-exchange chromatography.

Monoclonal antibody against a peptide of the present invention may be obtained by fusing cells of an immortalising cell line with cells which produce antibody against the viral or topographically related peptide, and culturing the fused immortalised cell line. Typically, a non-human mammalian host, such as a mouse or rat, is inoculated with the peptide. After sufficient time has elapsed for the host to mount an antibody response, antibody producing cells, such as the splenocytes, are removed. Cells of an immortalising cell line, such as a mouse or rate myeloma cell line, are fused with the antibody producing cells and the resulting fusions screened to identify a cell line, such as hybridoma, that secretes the desired monclonal antibody. The fused cell line may be cultured and the monoclonal antibody purified from the culture media in a similar manner to the purification of polyclonal antibody.

Diagnostic assays based upon the present invention may be used to determine the presence or absence of HCV infection. They may also be used to monitor treatment of such infection, for example in interferon therapy.

In an assay for the diagnosis of viral infection, there are basically three distinct approaches that can be adopted involving the detection of viral nucleic acid, viral antigen or viral antibody. Viral nucleic acid is generally regarded as the best indicator of the presence of the virus itself and would identify materials likely to be infectious. However, the detection of nucleic acid is not usually as straightforward as the detection of antigens or antibodies since the level of target can be very low. Viral antigen is used as a marker for the presence of virus and as an indicator of infectivity. Depending upon the virus, the amount of antigen present in a sample can be very low and difficult to detect. Antibody detection is relatively straightforward because, in effect, the host immune system is amplifying the response to an infection by producing large amounts of circulating antibody. The nature of the antibody response can often be clinically useful, for example IgM rather than IgG class antibodies are indicative of a recent infection, or the response to a particular viral antigen may be associated with clearance of the virus. Thus the exact approach adopted for the diagnosis of a viral infection depends upon the particular circumstances and the information sought. In the case of HCV, a diagnostic assay may embody any one of these three approaches.

In an assay for the diagnosis of HCV involving detection of viral nucleic acid, the method may comprise hybridising viral RNA present in a test sample, or cDNA synthesised from such viral RNA, with a DNA sequence corresponding to the nucleotide sequence of SEQ ID NO: 1, 4 or 6 or encoding the peptide of formula I and screening the resulting nucleic acid hybrids to identify any HCV viral nucleic acid. The application of this method is usually restricted to a test sample of an appropriate tissue, such as a liver biopsy, in which the viral RNA is likely to be present at a high level. The DNA sequence corresponding to the nucleotide sequence of SEQ ID NO: 1, 4 or 6 or encoding the peptide of Formula I may take the form of an oligonucleotide or a cDNA sequence sequence optionally contained within a plasmid. Screening of the nucleic acid hybrids is preferably carried out by using a labelled DNA sequence. Preferably the peptide of the present invention is part of an oligonucleotide wherein the label is situated at a sufficient distance from the peptide so that binding of the peptide to the viral nucleic acid is not interfered with by virtue of the label being too close to the binding site. One or more additional rounds of screening of one kind or another may be carried out to characterise further the hybrids and thus identify any HCV viral nucleic acid. The steps of hybridisation and screening are carried out in accordance with procedures known in the art.

The present invention also provides a test kit for the detection of HCV viral nucleic acid, which comprises

i) a labelled oligonucleotide comprising a DNA sequence corresponding to the nucleotide sequence of SEQ ID NO: 1, 4 or 6 or encoding the peptide of formula I; and optionally;

ii) washing solutions, reaction buffers and a substrate, if the label is an enzyme.

Advantageously, the test kit also contains a positive control sample to facilitate in the identification of viral nucleic acid.

In an assay for the diagnosis of HCV involving detection of viral antigen or antibody, the method may comprise contacting a test sample with a peptide of the present invention or a polyclonal or monoclonal antibody against the peptide and determining whether there is any antigen-antibody binding contained within the test sample. For this purpose, a test kit may be provided comprising a peptide, as defined herein, or a polyclonal or monoclonal antibody thereto and means for determining whether there is any binding with antibody or antigen respectively contained in the test sample. The test sample may be taken from any of the appropriate tissues and physiological fluids mentioned above for the detection of viral nucleic acid. If a physiological fluid is obtained, it may optionally be concentrated for any viral antigen or antibody present.

A variety of assay formats may be employed. The peptide can be used to capture selectively antibody against HCV from solution, to label selectively the antibody already captured, or both to capture and label the antibody. In addition, the peptide may be used in a variety of homogeneous assay formats in which the antibody reactive with the peptide is detected in solution with no separation of phases.

The types of assay in which the peptide is used to capture antibody from solution involve immobilization of the peptide on to a solid surface. This surface should be capable of being washed in some way. Examples of suitable surfaces include polymers of various types (moulded into microtitre wells; beads; dipsticks of various types; aspiration tips; electrodes, and optical devices), particles (for example latex; stabilized red blood cells; bacterial or fungal cells; spores; gold or other metallic or metal-containing sols; and proteinaceous colloids) with the usual size of the particle being from 0.02 to 5 microns, membranes (for example of nitrocellulose; paper; cellulose acetate; and high porosity/high surface area membranes of an organic or inorganic material).

The attachment of the peptide to the surface can be by passive adsorption from a solution of optimum composition which may include surfactants, solvents, salts and/or chaotropes; or by active chemical bonding. Active bonding may be through a variety of reactive or activatible functional groups which may be exposed on the surface (for example condensing agents; active acid esters, halides and anhydrides; amino, hydroxyl, or carboxyl groups; sulphydryl groups; carbonyl groups; diazo groups; or unsaturated groups). Optionally, the active bonding may be through a protein (itself attached to the surface passively or through active bonding), such as albumin or casein, to which the viral peptide may be chemically bonded by any of a variety of methods. The use of a protein in this way may confer advantages because of isoelectric point, charge, hydrophilicty or other physico-chemical property. The viral peptide may also be attached to the surface (usually but not necessarily a membrane) following electrophoretic separation of a reaction mixture, such as immune precipitation.

After contacting (reacting) the surface bearing the peptide with a test sample, allowing time for reaction, and, where necessary, removing the excess of the sample by any of a variety of means, (such as washing, centrifugation, filtration, magnetism or capilliary action) the captured antibody is detected by any means which will give a detectable signal. For example, this may be achieved by use of a labelled molecule or particle as described above which will react with the captured antibody (for example protein A or protein G and the like; anti-species or anti-immunoglobulin-sub-type; rheumatoid factor; or antibody to the peptide, used in a competitive or blocking fashion), or any molecule containing an epitope contained in the peptide.

The detectable signal may be optical or radioactive or physico-chemical and may be provided directly by labelling the molecule or particle with, for example, a dye, radiolabel, electroactive species, magnetically resonant species or fluorophore, or indirectly by labelling the molecule or particle with an enzyme itself capable of giving rise to a measurable change of any sort. Alternatively the detectable signal may be obtained using, for example, agglutination, or through a diffraction or birefringent effect if the surface is in the form of particles.

Assays in which a peptide itself is used to label an already captured antibody require some form of labelling of the peptide which will allow it to be detected. The labelling may be direct by chemically or passively attaching for example a radio label, magnetic resonant species, particle or enzyme label to the peptide; or indirect by attaching any form of label to a molecule which will itself react with the peptide. The chemistry of bonding a label to the peptide can be directly through a moiety already present in the peptide, such as an amino group, or through an intermediate moiety, such as a maleimide group. Capture of the antibody may be on any of the surfaces already mentioned by any reagent including passive or activated adsorption which will result in specific antibody or immune complexes being bound. In particular, capture of the antibody could be by anti-species or anti-immunoglobulin-sub-type, by rheumatoid factor, proteins A, G and the like, or by any molecule containing an epitope contained in the peptide.

The labelled peptide may be used in a competitive binding fashion in which its binding to any specific molecule on any of the surfaces exemplified above is blocked by antigen in the sample. Alternatively, it may be used in a non-competitive fashion in which antigen in the sample is bound specifically or non-specifically to any of the surfaces above and is also bound to a specific bi- or poly-valent molecule (e.g. an antibody) with the remaining valencies being used to capture the labelled peptide.

Often in homogeneous assays the peptide and an antibody are separately labelled so that, when the antibody reacts with the recombinant peptide in free solution, the two labels interact to allow, for example, non-radiative transfer of energy captured by one label to the other label with appropriate detection of the excited second label or quenched first label (e.g. by fluorimetry, magnetic resonance or enzyme measurement). Addition of either viral peptide or antibody in a sample results in restriction of the interaction of the labelled pair and thus in a different level of signal in the detector.

A suitable assay format for detecting HCV antibody is the direct sandwich enzyme immunoassay (EIA) format. A peptide is coated onto microtitre wells. A test sample and a peptide to which an enzyme is coupled are added simultaneously. Any HCV antibody present in the test sample binds both to the peptide coating the well and to the enzyme-coupled peptide. Typically, the same peptide are used on both sides of the sandwich. After washing, bound enzyme is detected using a specific substrate involving a colour change. A test kit for use in such an EIA comprises:

(1) a peptide, as herein defined labelled with an enzyme;

(2) a substrate for the enzyme;

(3) means providing a surface on which a peptide is immobilised; and

(4) optionally, washing solutions and/or buffers.

It is also possible to use IgG/IgM antibody capture ELISA wherein an antihuman antibody is coated onto microlitre wells, a test sample is added to the well. Any IgG or IgM antibody present in the test sample will then bind to the anti-human antibody. A peptide of the present invention, which has been labelled, is added to the well and the peptide will bind to any IgG or IgM antibody which has resulted due to infection by HCV. The IgG or IgM antibody can be visualized by virtue of the label on the peptide.

It can thus be seen that the peptides of the present invention may be used for the detection of HCV infection in many formats, namely as free peptides, in assays including classic ELISA, competition ELISA, membrane bound EIA and immunoprecipitation. Peptide conjugates may be used in amplified assays and IgG/IgM antibody capture ELISA.

The peptide of the present invention may be incorporated into a vaccine formulation for inducing immunity to HCV in man. For this purpose the peptide may be presented in association with a pharmaceutically acceptable carrier.

For use in a vaccine formulation, the peptide may optionally be presented as part of an hepatitis B core fushion particle, as described in Clarke et al (Nature, 1987, 330, 381-384), or a polylysine based polymer, as described in Tam (PNAS, 1988, 85, 5409-5413). Alternatively, the peptide may optionally be attached to a particulate structure, such as liposomes or ISCOMS.

Pharmaceutically acceptable carriers include liquid media suitable for use as vehicles to introduce the peptide into a patient. An example of such liquid media is saline solution. The peptide may be dissolved or suspended as a solid in the carrier.

The vaccine formulation may also contain an adjuvant for stimulating the immune response and thereby enhancing the effect of the vaccine. Examples of adjuvants include aluminium hydroxide and aluminium phosphate.

The vaccine formulation may contain a final concentration of peptide in the range from 0.01 to 5 mg/ml, preferably from 0.03 to 2 mg/ml. The vaccine formulation may be incorporated into a sterile container, which is then sealed and stored at low temperature, for example 4° C., or may be freeze-dried.

In order to induce immunity in man to HCV, one or more doses of the vaccine formulation may be administered. Each dose may be 0.1 to 2 ml, preferably 0.2 to 1 ml. A method for inducing immunity to HCV in man, comprises the administration of an effective amount of a vaccine formulation, as hereinbefore defined.

The present invention also provides the use of a peptide as herein defined in the preparation of a vaccine for use in the induction of immunity to HCV in man.

Vaccines of the present invention may be administered by any convenient method for the administration of vaccines including oral and parenteral (e.g. intravenous, subcutaneous or intramuscular) injection. The treatment may consist of a single dose of vaccine or a plurality of does over a period of time.

In the sequence listing, there are listed SEQ ID NO: 1 to 29 to which reference is made in the description and claims.

The embodiments of the invention will now be illustrated.

### EXAMPLE 1

### Identification of Epitopic Domain of the HCV Core Protein

The N-terminal 97 amino acid residues of HCV Core protein SEQ ID NO: 8 was mapped with PEPSCAN analysis. This process involved the serological analysis of sequentially overlapping peptides which spanned the region of interest, to identify epitopic domains. Ninety overlapping octameric peptides were synthesized on polypropylene pin supports according to the manufacturer's instructions (Cambridge Research Biochemicals), such that each peptide overlapped with the preceding sequence by the seven N-terminal residues, and with the subsequent peptide by the seven C-terminal residues. Serum or IgG purified from the serum of individuals confirmed to be seropositive for HCV were used to screen the peptides.

### EXAMPLE 2

### Screening of the Peptides

The assay procedure followed was performed basically as described in the manufacturers instruction manual. The time and temperature of the test antibody incubation differed from that prescribed, thereby decreasing the time taken for each assay.

Sera: A total of 16 sera were assayed by PEPSCAN. These consisted of 12 sera from paid U.S. Donors which were identified as being positive for antibodies against HCV in routine screening at the site of donation. The remaining four sera were from patients diagnosed as having non-A non-B hepatitis, and which were subsequently shown to contain antibodies to HCV. Sera were screened either as the crude serum fraction, or as a caprylic acid preparation. Caprylic acid purification of IgG from the sera was performed as described by Stienbuch and Audran (1969) Arch. Biochem. Biophys. 134, 279-284.

Assay: The assay format involves sequentially incubating the various pins in microwell plates containing blocking, test antibody, conjugate, and substrate solutions. The immune complex builds up on the pin with the enzyme labelled conjugate being immobilized on the bound anti-peptide antibody from the test serum. When the pin is then immersed in substrate, the enzyme brings about the colour change which is used to quantify the amount of anti-peptide antibody bound.

Blocking: Pins are immersed in 200 μl of Supercocktail (1% Ovalbumin, 1% bovine serum albumin in 0.15M phosphate buffered saline pH 7.2—0.1% Tween 20) and left to stand at room temperature for 1 hour.

Test antibody: The test antibody is diluted between 1/200 (IgG) and 1/1000 (serum) in supercocktail. The pins are then stood in this solution (175 μl/well) at 37° C. for 1 hour. After this incubation the pins are washed four times in 0.15M phosphate buffered saline pH 7.2—0.05% Tween 20, each at room temperature for 10 minutes.

Conjugate: Rabbit anti-human IgG coupled to horseradish peroxidase is diluted 1/1000 in supercocktail. The peptide bearing pins are then stood in this solution at room temperature for 1 hour. Once again the pins are then washed four times as above.

Substrate: The substrate solution is made up fresh for each assay. To 100 ml of 0.1M phosphate, 0.08M citrate buffer pH 4.0 is dissolved 50 mg of 2,2′-AZINO-bis (3-ethylbenzthiazo-linsulphonic acid (ABTS) and 0.3 μl/ml 30% hydrogen peroxide is added. The pins are immersed in the wells containing substrate solution (150 μl/well) and incubated at room temperature in the dark. Once sufficient colour has developed the reaction is stopped by removing the pins. The plates are then read immediately in a spectrophotometric plate reader at a wavelength of 405 nm. The absorbance values are then plotted against pin number. The epitopic domains are then defined as the continuous sequence of amino acids common to all positive pins in the domain i.e.

| | | |
|---|---|---|
| Positive Pin 1 | Gly-Val-Tyr-Leu-Leu-Pro-Arg-Arg | (SEQ ID NO: 9) |
| | ( G V Y L L P R R) | |
| Positive Pin 2 | Val-Tyr-Leu-Leu-Pro-Arg-Arg-Gly | (SEQ ID NO: 10) |
| | ( V Y L L P R R G) | |
| Positive Pin 3 | Tyr-Leu-Leu-Pro-Arg-Arg-Gly-Pro | (SEQ ID NO: 11) |
| | ( Y L L P R R G P) | |

In this example the epitope has the sequence Tyr-Leu-Leu-Pro-Arg-Arg (YLLPRR) (SEQ ID NO's: 1 and 2).

Cleaning: After use the pins are cleaned by ultrasonication in disruption buffer (1% sodium dodecyl sulphate, 0.1% 2-mercaptoethanol in 0.1M sodium dihydrogen phosphate) heated to 60° C. Pins are sonicated for 30 minutes and then washed twice in distilled water at 60° C. and once in boiling methanol. Pins can then be stored at room temperature sealed in bags containing silica gel, or can be re-used immediately for the next assay.

Several domains within the N-terminal region of HCV Core protein were thus identified as being epitopic. One of these regions, the immunodominant epitope, was detected to a greater or lesser extent by all of the sera tested. The immunodominant epitope is situated from residues 35-40 of the Core protein having the amino acid sequence Tyr-Leu-Leu-Pro-Arg-Arg (YLLPRR) SEQ ID NO's 1 and 2. Sample assay results are shown in Table 1.

TABLE 1
Absorbance Units
for different sera

| | SEQ ID | | | | | |
|---|---|---|---|---|---|---|
| Peptide | NO | 65221 | 65241 | 65276 | 65234 | L |
| GGGQIVGG | 12 | 137 | 121 | 127 | 134 | 121 |
| GGQIVGGV | 13 | 323 | 122 | 119 | 179 | 119 |
| GQIVGGVY | 14 | 216 | 112 | 128 | 165 | 138 |
| QIVGGVYL | 15 | 329 | 125 | 165 | 152 | 161 |
| IVGGVYLL | 16 | 268 | 126 | 183 | 186 | 158 |
| VGGVYLLP | 17 | 83 | 144 | 132 | 235 | 169 |
| GGVYLLPR | 18 | 132 | 311 | 246 | 344 | 191 |
| GVYLLPRR | 19 | 613 | 511 | 350 | 229 | 378 |
| VYLLPRRG | 20 | 769 | 641 | 440 | 280 | 306 |
| YLLPRRGP | 21 | 332 | 367 | 248 | 177 | 242 |
| LLPRRGPR | 22 | 142 | 129 | 98 | 101 | 133 |
| LPRRGPRL | 23 | 97 | 94 | 139 | 145 | 148 |
| PRRGPPLG | 24 | 85 | 73 | 129 | 109 | 110 |
| RRGPRLGV | 25 | 145 | 85 | 150 | 146 | 123 |
| RGPRLGVR | 26 | 168 | 94 | 167 | 99 | 132 |
| GPRLGVRA | 27 | 111 | 60 | 146 | 75 | 101 |
| PRLGVRAT | 28 | 166 | 79 | 155 | 88 | 115 |
| RLGVRATR | 29 | 81 | 180 | 177 | 100 | 124 |

The amino acid residues are denoted by the single letter code (Eur. J. Biochem. 138, 9-37, 1984). The underlined characters represent the immunodominant region.

### EXAMPLE 3

### Preparation of Novel Mimic of Original Epitope

The Replacement Net Method was used to sequentially replace each of the constituent amino acid residues in the epitope with each of the other genetically encoded amino acids.

For Replacement Net analysis, a parent sequence is entered into the Epitope Mapping Software provided by Cambridge Research Biochemicals, and a set of replacement peptides is generated. This process was performed for the six amino acid residue immunodominant core epitope of HCV. The peptides were synthesized on polypropylene pins as directed by the manufacturer (CRB). The Assay format was identical to that followed for PEPSCAN above.

The Replacement Net of the core immunodominant epitope sequence Tyr-Leu-Leu-Pro-Arg-Arg (YLLPRR) (SEQ ID NO: 1 and 2) with the 12 paid donor human sera as described above was considered.

For each position within the epitope, the frequency of each replacement residue as an acceptable replacement for the parent sequence is given. Replacement residues were deemed acceptable when the signal of the new peptide was 80% of the parent sequence or greater.

Based on these results, the substitute amino acid residues at each position, which are most likely potentiating substitutions, can be obtained. These are shown in Table 2. The residue in italics is the parent sequence.

TABLE 2

| | | Frequency of relative signal strength (n = 12) | | | | |
|---|---|---|---|---|---|---|
| Parent | Amino | High- | | | | |
| Residue | Acid | est | 2nd-5th | 6th-10th | Below 10th | All |
| 1-Tyrosine | D | 1 | 8 | 1 | 0 | 10 |
| | E | 2 | 8 | 1 | 0 | 11 |
| | I | 0 | 3 | 7 | 0 | 10 |
| | L | 7 | 1 | 1 | 0 | 9 |
| | Y | 2 | 3 | 5 | 1 | 11 |
| 2-Leucine | C | 2 | 8 | 1 | 0 | 11 |
| | D | 6 | 4 | 0 | 0 | 10 |
| | E | 1 | 6 | 4 | 0 | 11 |
| | L | 1 | 2 | 3 | 2 | 8 |
| 3-Leucine | L | 2 | 0 | 1 | 0 | 3 |
| | V | 6 | 5 | 0 | 0 | 11 |
| 4-Proline | M | 4 | 5 | 0 | 0 | 9 |
| | P | 3 | 1 | 1 | 0 | 5 |
| 5-Arginine | C | 0 | 8 | 2 | 0 | 10 |
| | D | 4 | 5 | 1 | 1 | 11 |
| | E | 2 | 4 | 3 | 1 | 10 |
| | F | 0 | 2 | 7 | 3 | 12 |
| | M | 0 | 2 | 2 | 6 | 10 |
| | N | 0 | 0 | 6 | 5 | 11 |
| | P | 1 | 5 | 4 | 1 | 11 |
| | Q | 2 | 6 | 2 | 0 | 10 |
| | R | 0 | 2 | 1 | 7 | 10 |
| 6-Arginine | C | 0 | 4 | 0 | 6 | 10 |
| | D | 3 | 2 | 3 | 2 | 10 |
| | E | 1 | 6 | 2 | 1 | 10 |
| | R | 2 | 1 | 5 | 3 | 11 |
| | S | 3 | 5 | 1 | 1 | 10 |

### EXAMPLE 4

### Preparation of AC 22

Fully protected peptide-resin was assembled on a p-hydroxymethylphenoxymethyl (HMP) resin by stepwise solid phase synthesis (proceeding from the carboxyl terminal residue towards the amino terminal residue) according to the general procedure set out in Applied Biosystems User Bulletin No 33, November 1990. HMP resin was transferred into the reaction vessel of an Applied Biosystems Peptide Synthesizer Model 431A. The carboxyl-terminal amino acid residue was coupled to the resin using the standard Applied Biosystems Fastmoc cycle protocol. Unreacted coupling sites on the resin were then blocked by capping with benzoic anhydride. All subsequent amino acids were then added in a stepwise manner moving from the carboxyl terminus to the amino terminus using the Fastmoc cycle protocol. The N-α-amino group of all peptides was protected by the 9-fluorophenylmethoxycarbonyl (Fmoc) linkage. Side chain functional groups of the relevant amino acids were protected by the following groups

Cys-Trt (Trityl)

Gly-Trt (Trityl)

Tyr-tBu (Tert. Butyl)

Arg-Pmc (2,2,5,7,8-pentamethylchroman-6-sulphonyl)

The fully protected peptide-resin was transferred to a pear-shaped flask and treated with ethanedithiol (0.25 ml), thioanisole (0.5 ml), water (0.5 ml) and TFA (8.75 ml). The mixture was stirred for 90 minutes at room temperature, then filtered through glass wool in a Pasteur pipette. The filtrate was dropped into ice-cold ether, contained in a Corex centrifuge tube, whereupon the peptide precipitated. The tube was centrifuged and the liquor aspirated to leave the peptide pellet in the tube. The peptide was washed with ether (using a spatula to dis-aggregate the pellet and the peptide recovered by centrifugation. This was repeated a total of six times. The peptide was finally dried by centrifugation in vacuo.

### EXAMPLE 5

### Vaccine Formulation

A vaccine formulation may be prepared by conventional techniques using the following constituents in the indicated amounts.

| | | |
|---|---|---|
| HCV viral peptide | >0.36 | mg |
| (whether alone, as a longer | | |
| polypeptide or with a cocktail | | |
| or other peptides) | | |
| Thiomersal | 0.04-0.2 | mg |
| Sodium Chloride | <8.5 | mg |
| Water | <1 | ml |

1. A HCV viral peptide having the sequence:
Tyr-Leu-Leu-Pro-Arg-Arg (SEQ ID NO's: 1 and 2)

2. A peptide having the sequence of the general formula I
X₁—X₂—X₃—X₄—X₅—X₆ (SEQ ID NO: 3) I
wherein;
X₁ represents Tyrosine, Aspartic Acid, Glutamic Acid, Leucine or Isoleucine;
X₂ represents Leucine, Cysteine, Aspartic Acid or Glutamic Acid;
X₃ represents Leucine, Valine;
X₄ represents Proline or Methionine;
X₅ represents Arginine, Aspartic Acid, Glutamic Acid, Proline, Cysteine, Phenylalanine, Isoleucine, Asparagine, Glutamine or Methionine; and
X₆ represents Arginine, Aspartic Acid, Glutamic Acid, Serine or Cysteine
and wherein the peptide is not of the formula Tyr-Leu-Leu-Pro-Arg-Arg (SEQ ID NO: 1 and 2).

3. A viral peptide having as set forth in SEQ ID NO's: 4 and 5 or SEQ ID NO's: 6 and 7.

4. A DNA sequence encoding a peptide as claimed in claim 1, 2 or 3.

5. A DNA sequence as claimed in claim 4 as set forth in SEQ ID NO: 1, 4 or 6.

6. An expression vector containing a DNA sequence as claimed in claim 4 or 5 being capable in an appropriate host of expressing the DNA sequence to produce a peptide.

7. A host cell transformed with an expression vector as claimed in claim 6.

8. A process for preparing a peptide as claimed in any of claims 1 to 3 which comprises isolating the DNA sequence, as set forth in SEQ ID NO: 1, 4 or 6, from the HCV genome, or synthesising a DNA sequence encoding the peptide, as set forth in SEQ ID NO: 1, 4, 6, or generating a DNA sequence encoding for the peptide of Formula I, inserting the DNA sequence into an expression vector such that it is capable, in an appropriate host, of being expressed, transforming an host cell with the expression vector, culturing the transformed host cell, and isolating the peptide.

9. A process for preparing the peptide as claimed in any of claims 1 to 3 characterized in that it is prepared synthetically.

10. A polyclonal or monoclonal antibody against a peptide as claimed in any of claims 1 to 3.

11. A method of the detection of HCV viral nucleic acid which comprises hybridizing viral RNA present in the test sample or cDNA synthesized from such RNA, with a DNA sequence corresponding to SEQ ID NO: 1, 4, 6, or encoding the peptide of formula I and screening the resulting nucleic acid hybrids to identify any HCV viral nucleic acid.

12. A method for the detection of HCV viral antibody which comprises contacting a test sample with a peptide as claimed in any of claims 1 to 3 or a polyclonal or monoclonal antibody as claimed in claim 10 and determining whether there is any antigen-antibody binding contained within the test sample.

13. A test kit for the detection of HCV viral antibody which comprises the peptide as claimed in any of claims 1 to 3 or a polyclonal or monoclonal antibody as claimed in claim 10 and means for determining whether there is any antigen-antibody binding contained in the test sample.

14. A vaccine formulation which comprises a peptide as claimed in any of claims 1 to 3 in association with a pharmaceutically acceptable carrier.

15. A method for inducing immunity in man to HCV which comprises the administration of an effective amount of a vaccine formulation according to claim 14.
